(19) United States
(12) Patent Application Publication
(10) Pub. No.: US 2025/0135097 A1
(43) Pub. Date: May 1, 2025

(54) SECURE FLUID CONNECTOR

(71) Applicant: Fresenius Medical Care Holdings, Inc., Waltham, MA (US)

(72) Inventor: Kevin Nye, Seymour, TN (US)

(21) Appl. No.: 18/497,234

(22) Filed: Oct. 30, 2023

(51) Int. Cl. A61M 5/14; A61M 39/10

(52) U.S. Cl. CPC A61M 5/1413; A61M 39/1011; A61M 2039/1033; A61M 2039/1044

(57) ABSTRACT

In one aspect a medical fluid connector, includes: a first end including an opening configured to allow fluid flow into the medical fluid connector; a second end including an opening configured to allow fluid flow out of the medical fluid connector; an interior volume forming a flow path extending from the first end to the second end; a frangible block forming a first seal in the flow path at a location between the first end and the second end, the frangible block configured to be broken to break the first seal and allow fluid flow around the frangible block; and a flexible seal forming a second seal in the flow path at a location between the frangible block and the second end.

### TECHNICAL FIELD

This disclosure relates to fluid connectors, and more particularly to medical fluid connectors such as used in connection with dialysis treatments.

### BACKGROUND

Medical devices, such as dialysis machines, are known for use in the treatment of renal disease. The two principal dialysis methods are hemodialysis (HD) and peritoneal dialysis (PD). During hemodialysis, the patient's blood is passed through a dialyzer of a hemodialysis machine while also passing dialysate through the dialyzer. A semi-permeable membrane in the dialyzer separates the blood from the dialysate within the dialyzer and allows diffusion and osmosis exchanges to take place between the dialysate and the blood stream. During peritoneal dialysis, the patient's peritoneal cavity is periodically infused with dialysate, or dialysis solution. The membranous lining of the patient's peritoneum acts as a natural semi-permeable membrane that allows diffusion and osmosis exchanges to take place between the solution and the blood stream. Automated peritoneal dialysis machines, also called PD cyclers, are designed to control the entire peritoneal dialysis process so that it can be performed at home, usually overnight, without clinical staff in attendance. Both HD and PD machines may include displays with touch screens or other user interfaces that display information of a dialysis treatment and/or enable an operator or patient to interact with the machine.

Dialysis machines may have a disposable set which has several connectors and tubing, also referred to herein as lines, used in connection with the dialysis treatment and through which medical fluid flows during the dialysis treatment. Home dialysis patients are required to make multiple tubing connections in the process or setting up their home dialysis machine and treatment.

With some technologies, users perform a multi-step process to connect fluid tubes for flow. For example, with current PD solution bag connectology, a user (e.g., a patient or caregiver) first secures a fluid connection between a PD solution bag and fluid delivery set attached to a PD machine by connecting two tube end connectors. After this initial connection is made, the user manually breaks a frangible plastic component, which may be referred to a as a cone in some products. This breaking of the frangible component allows the fluid to flow from the solution bag into and through the connected tubing connectors to the fluid delivery set. In some cases, users with weak hands or arthritis have difficulty breaking the cone. A tool is available, but some users will use household tools (e.g., a wrench or pliers) to break the frangible cone and make the connection. In some cases, users will attempt to make the connection but do not notice it is incomplete. This can cause alarms and delays in treatments. Moreover, the presence of the broken-off frangible cone can in some circumstances impede solution flow during the course of treatment.

Accordingly, it would be desirable to provide a connector system that reliably and securely forms a fluid-tight flow path, while also simplifying the process of breaking the fluid seal (e.g., frangible cone) of the fluid container.

### SUMMARY

This disclosure connectors for forming fluid connections for medical fluids.

In one aspect, a medical fluid connector, includes: a first end including an opening configured to allow fluid flow into the medical fluid connector; a second end including an opening configured to allow fluid flow out of the medical fluid connector; an interior volume forming a flow path extending from the first end to the second end; a frangible block forming a first seal in the flow path at a location between the first end and the second end, the frangible block configured to be broken to break the first seal and allow fluid flow around the frangible block; and a flexible seal forming a second seal in the flow path at a location between the frangible block and the second end.

In some examples, the medical fluid connector further includes a guideway configured to guide a mating connector into a coupled position relative to the medical fluid connector, the guideway spanning a distance along an axis defined by the flow path that is greater than a distance between the frangible block and the flexible seal. In some examples, the guideway is helical.

In some examples, the flexible seal is an elastomeric membrane. In some examples, the elastomeric membrane includes at least one of (a) a slit, (b) a perforation, or (c) a weakened region.

In some examples, the medical fluid connector further includes a helical guideway configured to guide a mating connector into a coupled position relative to the medical fluid connector, the helical guideway sweeping a quarter turn about an axis defined by the flow path, wherein the quarter turn of the guideway spans a distance along the axis that is greater than a distance between the frangible block and the flexible seal. In some examples, the helical guideway is a thread or a groove.

In one aspect, a medical fluid container may include the medical fluid connector and a fluid reservoir in fluid communication with the flow path of the medical fluid connector.

In some examples, the medical fluid container further includes a volume of medical fluid disposed in the fluid reservoir.

In one aspect, a medical fluid connector set includes the medical fluid connector and a mating connector configured to plug into the medical fluid connector such that the first seal and the second seal are broken to allow fluid to flow through the flow path from the first end into the second end and into the mating connector.

In some examples, the medical fluid connector of the medical fluid connector set includes a helical guideway, and the mating connector is configured to engage with and progress along the helical guideway such that a quarter turn of the mating connector relative to the medical fluid connector causes, sequentially, (a) penetration of the flexible seal to break the second seal and (b) breaking of the frangible block to break the first seal.

In some examples, the mating connector includes a rigid protrusion configured to penetrate the flexible seal and break the frangible block when the second connector is progressed along the helical guideway.

In some examples, the rigid protrusion is hollow such that fluid is allowed to flow from the first end of the medical fluid connector through the rigid protrusion when the rigid protrusion penetrates the flexible seal and breaks the frangible block.

In some examples, the medical fluid connector set includes a latch configured to lock the medical fluid connector and the mating connector in position after the quarter turn to prevent disengagement of the medical fluid connector and the mating connector.

In some examples, the medical fluid connector and the mating connector are configured to provide at least one of (a) auditory feedback and (b) tactile feedback to indicate a complete connection of the medical fluid connector and the mating connector.

In some examples, the medical fluid connector and the mating connector are configured such that the at least one of (a) auditory feedback and (b) tactile feedback are generated by a latching engagement between the medical fluid connector and the mating connector.

In one aspect, a method includes: joining a first medical fluid connector to a second medical fluid connector; and rotating the first and second medical fluid connectors relative to each other, wherein the rotation causes, sequentially, a flexible seal to be penetrated and a frangible block to be broken to allow fluid flow between the first and second medical fluid connectors.

In some examples, the rotation is a quarter turn of the first medical fluid connector relative to the second medical fluid connector.

In some examples, the method also includes latching the first and second medical fluid connectors against reverse rotation after penetration of the flexible seal and breaking of the frangible block.

In one aspect, a medical fluid set includes: a first connector; and a second connector configured to mechanically engage the first connector such that a single rotation of the first connector relative to the second connector causes sequentially (a) opening of a flexible seal and (b) breaking of a frangible seal to form a hermetically sealed flow path between the first connector and the second connector.

In some examples, the single rotation is a quarter turn of the first connector relative to the second connector.

In some examples, the flexible seal is part of the first connector, and the second connector comprises a rigid protrusion configured to penetrate and open the flexible seal during the single rotation to form a hermetic seal between the flexible seal and the rigid protrusion.

In some examples, the rigid protrusion includes an inner lumen configured to allow fluid flow as part of the hermetically sealed flow path.

In some examples, the first connector also includes a frangible block forming the frangible seal, the frangible block having (a) an attached state wherein the frangible block forms a hermetic seal preventing fluid flow through the first connector and (b) a detached state in which the frangible block is separated from the remainder of the first connector to allow fluid flow through the first connector.

In some examples, the rigid protrusion is also configured to, during the single rotation, contact the frangible block to convert the block from the attached state to the detached state. In some examples, the single rotation is a quarter turn of the first connector relative to the second connector.

In some examples, the single rotation is about an axis that is parallel to a flow path through tubing set.

Other aspects, features, and advantages will be apparent from the description, the drawings, and the claims.

### DESCRIPTION OF DRAWINGS

FIG. 1 is a side view of a portion of a fluid line set in a disconnected state.

FIG. 2 is a side view of the fluid line set of FIG. 1 in a connected state.

FIG. 3 is a cross sectional view of the fluid line set of FIG. 1 in a disconnected state.

FIG. 4 is a cross sectional view of the portion of the fluid line set of FIG. 1 in the connected state.

FIG. 5 is a perspective view of a frangible block of the fluid line set of FIG. 1.

FIG. 6 is a cross sectional view of the portion of the fluid line set of FIG. 1 in the connected state with flow lines.

FIG. 7 is a perspective view of a male portion of a connector of the fluid line set of FIG. 1.

FIG. 8 is a cross sectional view of a locking mechanism of the fluid line set of FIG. 1 in an unlocked state.

FIG. 9 is a cross sectional view of the locking mechanism of FIG. 8 in a locked state.

Like reference symbols in the various drawings indicate like elements.

### DETAILED DESCRIPTION

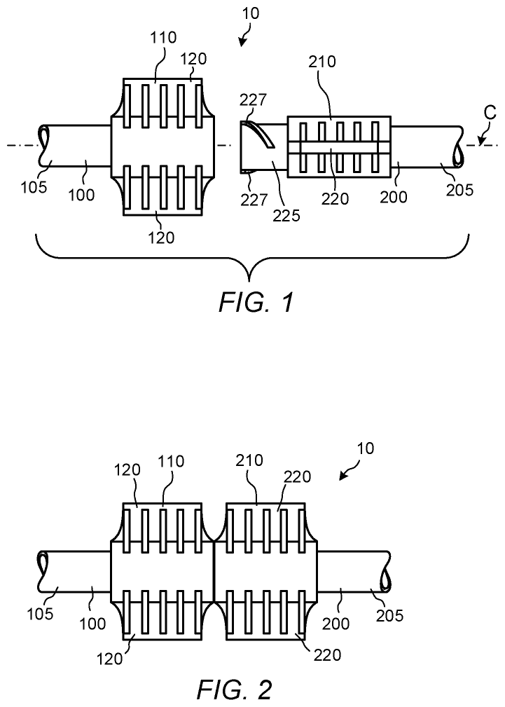

FIG. 1 shows a portion of a fluid line set 10. The fluid line set 10 includes a connection set that includes a first line segment 100 and a second line segment 200. First line segment 100 includes a first connector 110 attached to a flexible tubular fluid line 105. Likewise, second line segment 200 includes a second connector 210 attached to a flexible tubular fluid line 205. When the connectors 110 and 210 are fully attached, as shown in FIG. 2 and as described in greater detail below, a fluid-tight connection is formed to allow the flow of fluid from interior lumen of the first fluid line 105 to the interior lumen of the second fluid line 205.

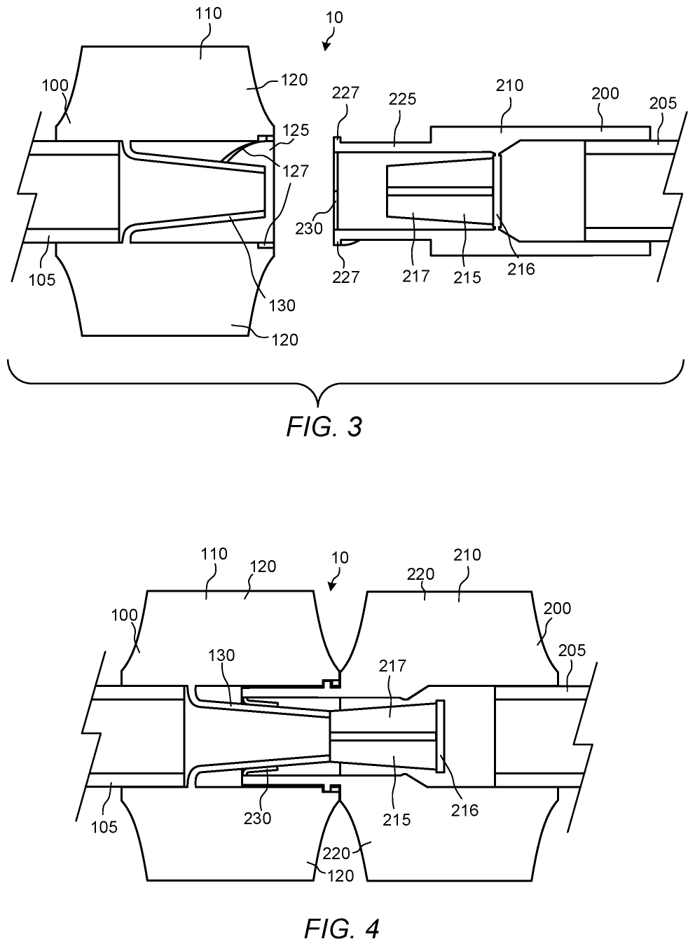

As shown in FIG. 1 and the cross-sectional view of FIG. 3, the line set 10 is in an initial, disconnected state. In this state, the first and second line segments 100 and 200 are physically separate and there is no fluid connection between the two fluid lines 105 and 205. Moreover, referring to FIG. 3, the second line segment 200 is capped off by a frangible block 215, which forms a hermetic seal with respect to liquid inside the second line segment 200. To facilitate illustration of the geometry of the frangible block 215 of this example, the block 215 is shown without being split in cross-section along the line A-A of FIG. 1, whereas the remainder of the connector 210 is.

To connect the line segments 100 and 200, the user orients the connectors 110 and 210 as shown in FIG. 1. To facilitate orientation, improve grip, and ease required twisting force, the connectors 110 and 210 include wings 120, 220. In the illustrated example, the initial position entails orienting the wings 220 at a right angle relative to the wings 110, the subsequent rotation being about the axis C of the flow path through the connectors.

In the initial position of FIGS. 1 and 3, a set of external threads 227 on a threaded projection 225 of the connector 210 aligns with a corresponding set of internal threads or channels 127 of the connector 110, so that when the two connectors 110 and 210 are pressed together, the threads 227 and channels 127 engage each other to allow the connectors 110, 210 to rotated relative to each other to complete the liquid tight fluid connection, which is shown in FIGS. 2 and 4. Although the threaded connection of the example line set 10 are the in form of a thread and a channel, it should be understood that any suitable thread members or other threaded engagement mechanism may be provided. For example, some examples may have thread projections instead of channels on the interior surface of the first connector 110.

The channels 127 and threads 227 are dimensioned such that a quarter turn of the connectors 110 and 210 relative to each other draws the connectors into the fully engaged position shown in FIGS. 2 and 4. To allow the connectors to engage properly even if one is rotated 180 degrees two pairs of mating channels 127 and threads 227 are provided, spaced apart 180 degrees from each other about the axis about which the connectors 110 and 210 are twisted to form the connection. Since the quarter turn brings the wings 120, 220 into alignment with each other, the user has a clear indication that the connection is complete. In should be understood, however, that in other examples, there may be only a single thread or more than two threads.

Referring to FIG. 3, the first connector 110 includes a rigid internal protrusion 130 in the interior space of the first connector 110. The internal protrusion 130 includes an internal lumen through which the fluid flows when the connection is complete.

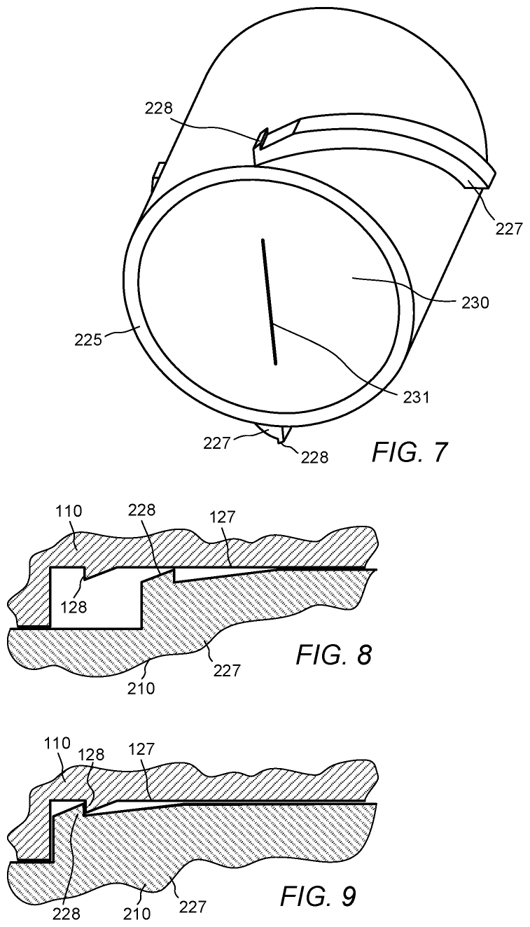

In addition to forming part of the flow path, the protrusion performs two additional functions as a result of the quarter-turn connection. In particular, the protrusion 130 presses into a split elastomeric (e.g., silicone) disk 230 of the second connector 210. This causes the disk 230 to stretch and allows the protrusion 130 to extend through and beyond the opening created at the split. Since, as shown in the connected position in FIG. 4, the elastomeric disk 230 is stretched tightly around the entire periphery of the protrusion 130, it forms a hermetic seal between the first and second connectors 110 and 210 to allow leak-free flow therethrough. Referring to FIG. 7, the split is in the form of a linear cut or slit 231. It should be understood, however, that in other examples, the split may be non-linear, multi-sectioned, or a single point and may be either a through-perforation, partial-perforation, or weakened location relative to the remainder of the disk 230.

The other function of the protrusion 130 as part of the quarter-turn motion is to contact and break off the frangible block 215 to break the seal of the second connector 210, thereby allowing fluid to flow in either direction through the connection.

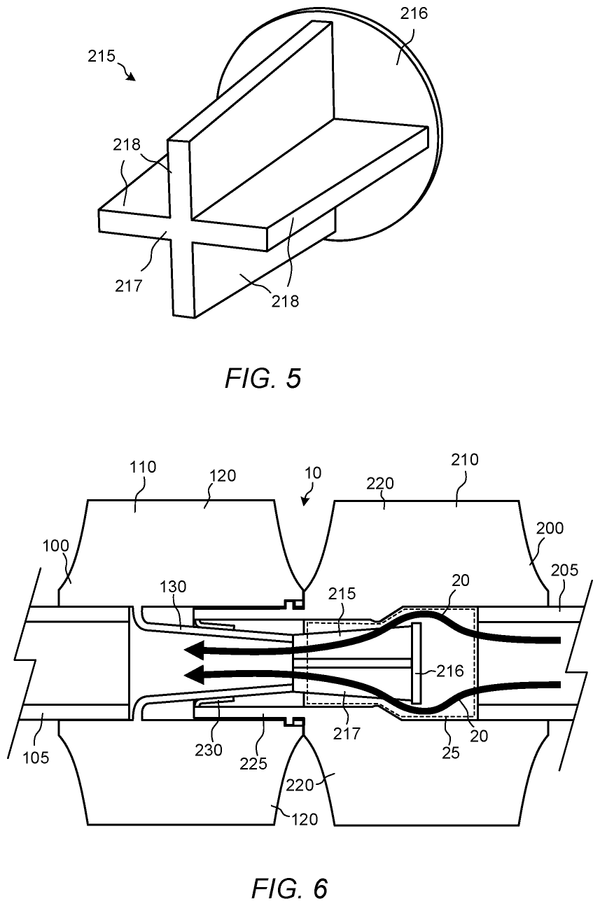

Referring to FIG. 5, the frangible block 215 includes a circular base 216 from which a ribbed extension 217 projects. The solid circular base allows the frangible block 215 to be frangibly connected to the remainder of the connector 210 at the outer periphery of the block 215, as shown in FIG. 3.

The ribs 218 of the extension 217 have multiple functions. For breaking the seal, they give the extension 217 mechanical strength to transfer the axial pushing force from the internal protrusion 130 of the first connector 110 to the base 216 to ensure breakage of the block 215 from the body of the connector 210. In this regard, the extension 217 and the internal protrusion 130 each has a respective face that is perpendicular to the axis along which the connection is made. This facilitates keeping the block 215 in its axial alignment during breakage to encourage a clean break.

Referring to FIG. 6, a further function of the ribs 217 is to allow flow paths, illustrated by arrows 20, around the frangible block 215 after it has been broken free. Because the frangible connection has a diameter that is smaller than the diameter of the internal flow path of the connector 210 in the region where the circular base 216 resides after being pushed by the protrusion 130, a flow path is created between the circular base 216 and the interior wall of the connector 210, thereby allowing bi-directional flow in the space between the outer periphery of the base 216 and the wall. The fluid, as shown by the arrows 20, then flows along the ribbed extension 217. The ribbed geometry allows for fluid flow between the ribs 218 to reduce drag and flow resistance.

After the frangible block 215 is broken free from the body of the connector 210, it is positioned within a constrained space 25 corresponding approximately to the space bounded by the dashed line of FIG. 6. In this example, the frangible block 215 may move axially, move radially, rotate, and/or pivot freely but limited by the bounds of the constrained space 25. Due to the geometry of both the constrained space 25 and the frangible block 215, adequate flow is maintained regardless of the orientation of the frangible block 215 relative to the constrained space 25 within the main body of the connector 210. In other examples, the frangible block may be substantially inhibited from moving axially, moving radially, rotating, and/or pivoting relative to the constrained space 25 when the connectors 110 and 210 are in the fully joined position of FIGS. 2, 4, and 6.

In the illustrated example, the line set 10 has frangible block 215 and related features on the connector 210 that has the male-threaded projection 225, and the internal protrusion 130 and related features on the connector 210 with interior-threaded features. However, in other examples, these aspects may be reversed, such that the frangible block 215 and related features are part of the connector with interior threaded features, the internal protrusion and related features are part of the connector with exterior or male threaded features.

Referring to FIGS. 7, 8, and 9, to prevent inadvertent disconnection of the connectors 110 and 210, some examples include a locking and feedback mechanism to provide an indication to a user that a complete connection has been made and to maintain the connectors in the connected position. In the illustrated example, the locking occurs via engagement of respective locking elements 128 and 228 in the form of projections. In this example, the first locking element 128 is a projection extending radially inwardly from the surface of the channel 127, and the second locking element is a projection extending outwardly from the thread 128. Referring to the cross-sectional views of FIGS. 8 and 9, as the connectors 110 and 210 move toward the fully engaged position, corresponding to the position of FIGS. 2, 4, 6, and 9, the second locking member 228 pushes past and engages the first locking member 128 to lock the connectors 110 and 210 in the fully engaged position. Although, this example utilizes a latching set of locking members disposed on the threads 227 and channels 127, it should be understood that any suitable securement mechanism may be provided at any suitable interface between the connectors 110 and 210.

To provide feedback to a user to indicate complete connection, the illustrated example provides both auditory and tactile feedback via the latching of the locking mechanisms. In particular, the sudden radial movement of the latching mechanisms as they pop into the latched positions of FIG. 9 provides an audible and tactile click to let the user know the connection is complete and secure, in addition to the visual feedback of the alignment of the wings 120 and 220. It should be understood that other examples may include auditory feedback without tactile feedback or tactile feedback without auditory feedback.

As used herein, an element or operation recited in the singular and preceded with the word “a” or “an” should be understood as not excluding plural elements or operations, unless such exclusion is explicitly recited. References to “one” embodiment or implementation of the present disclosure are not intended to be interpreted as excluding the existence of additional embodiments that also incorporate the recited features. Furthermore, a description or recitation in the general form of “at least one of [a], [b] or [c],” or equivalent thereof, should be generally construed to include [a] alone, [b] alone, [c] alone, or any combination of [a], [b] and [c].

Implementations of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A medical fluid connector, comprising:
a first end including an opening configured to allow fluid flow into the medical fluid connector;
a second end including an opening configured to allow fluid flow out of the medical fluid connector;
an interior volume forming a flow path extending from the first end to the second end;
a frangible block forming a first seal in the flow path at a location between the first end and the second end, the frangible block configured to be broken to break the first seal and allow fluid flow around the frangible block; and
a flexible seal forming a second seal in the flow path at a location between the frangible block and the second end.

2. The medical fluid connector of claim 1, further comprising a guideway configured to guide a mating connector into a coupled position relative to the medical fluid connector, the guideway spanning a distance along an axis defined by the flow path that is greater than a distance between the frangible block and the flexible seal.

3. The medical fluid connector of claim 2, wherein the guideway is helical.

4. The medical fluid connector of claim 1, wherein the flexible seal is an elastomeric membrane.

5. The medical fluid connector of claim 4, wherein the elastomeric membrane includes at least one of (a) a slit, (b) a perforation, or (c) a weakened region.

6. The medical fluid connector of claim 1, further comprising a helical guideway configured to guide a mating connector into a coupled position relative to the medical fluid connector, the helical guideway sweeping a quarter turn about an axis defined by the flow path, wherein the quarter turn of the guideway spans a distance along the axis that is greater than a distance between the frangible block and the flexible seal.

7. The medical fluid connector of claim 6, wherein the helical guideway is a thread or a groove.

8. A medical fluid container comprising:
the medical fluid connector of claim 1; and
a fluid reservoir in fluid communication with the flow path of the medical fluid connector.

9. The medical fluid container of claim 8, further comprising a volume of medical fluid disposed in the fluid reservoir.

10. A medical fluid connector set comprising:
the medical fluid connector of claim 1; and
a mating connector configured to plug into the medical fluid connector such that the first seal and the second seal are broken to allow fluid to flow through the flow path from the first end into the second end and into the mating connector.

11. The medical fluid connector set of claim 10, wherein the medical fluid connector includes a helical guideway and the mating connector is configured to engage with and progress along the helical guideway such that a quarter turn of the mating connector relative to the medical fluid connector causes, sequentially, (a) penetration of the flexible seal to break the second seal and (b) breaking of the frangible block to break the first seal.

12. The medical fluid connector set of claim 11, wherein the mating connector includes a rigid protrusion configured to penetrate the flexible seal and break the frangible block when the second connector is progressed along the helical guideway.

13. The medical fluid connector set of claim 12, wherein the rigid protrusion is hollow such that fluid is allowed to flow from the first end of the medical fluid connector through the rigid protrusion when the rigid protrusion penetrates the flexible seal and breaks the frangible block.

14. The medical fluid connector set of claim 11, further comprising a latch configured to lock the medical fluid connector and the mating connector in position after the quarter turn to prevent disengagement of the medical fluid connector and the mating connector.

15. The medical fluid connector set of claim 14, wherein the medical fluid connector and the mating connector are configured to provide at least one of (a) auditory feedback and (b) tactile feedback to indicate a complete connection of the medical fluid connector and the mating connector.

16. The medical fluid connector set of claim 15, wherein the medical fluid connector and the mating connector are configured such that the at least one of (a) auditory feedback and (b) tactile feedback are generated by a latching engagement between the medical fluid connector and the mating connector.

17. A method, comprising:
joining a first medical fluid connector to a second medical fluid connector; and
rotating the first and second medical fluid connectors relative to each other, wherein the rotation causes, sequentially, a flexible seal to be penetrated and a frangible block to be broken to allow fluid flow between the first and second medical fluid connectors.

18. The method of claim 17, wherein the rotation is a quarter turn of the first medical fluid connector relative to the second medical fluid connector.

19. The method of claim 17, further comprising latching the first and second medical fluid connectors against reverse rotation after penetration of the flexible seal and breaking of the frangible block.
